(19) United States
(12) Patent Application Publication
(10) Pub. No.: US 2016/0038712 A1
(43) Pub. Date: Feb. 11, 2016

(54) SOUND GENERATION SYSTEM WITH TWO AUDIO CHANNELS FOR TREATING PSYCHOLOGICAL/PSYCHIATRIC AND NEUROLOGICAL DISORDERS

(71) Applicant: Luca Gubert FINSTERLE, (US)

(72) Inventor: Luca Gubert Finsterle, Cernusco Sul Naviglio (IT)

(21) Appl. No.: 14/781,748

(22) PCT Filed: Apr. 3, 2014
(86) PCT No.: PCT/IB14/60397

§ 371 Date: Oct. 1, 2015

(30) Foreign Application Priority Data
Apr. 3, 2013 (IT) MI2013A000501

(51) Int. Cl. A61M 21/02

(52) U.S. Cl. CPC A61M 21/02; A61M 2021/0027

(57) ABSTRACT

A sound generation system with two audio channels for treating psychological/psychiatric and neurological disorders, comprising means (G/M, MD, DC, M/G MX) for generating two first audio signals, one for each audio channel (Ch1, Ch2), wherein each first signal is a random noise, and wherein the two first signals are not correlated to each other and define a relative reference sound level. The generation system comprises means (G/M, MD, DC, MX) for adding to both the first signals a second signal at a sound level having amplitude between +/−12 dB with respect to the relative reference sound level.

### FIELD OF THE INVENTION

The present invention relates to a sound generation system with two audio channels, designed to treat psychological/psychiatric and neurological disorders, such as sleep disorders/insomnia, anxiety, panic attacks, depression, obsessive-compulsive disorders, schizophrenia and neurological disorders such as headaches/migraines, tinnitus, diplopia, dementia, autism spectrum, Alzheimer's Disease and Pick's disease, as well as to increase physical and psychological peak performance in healthy subjects, producing an increase in neural cortical and top down/bottom up communication and a correlated loop deleting & resetting of dysfunctional neural patterns.

There are known audio systems for suppressing background noise, for improving the listener's perception of sound reproduction of the audio system. These systems have been developed for means of transport such as automobiles.

These audio systems are based on picking up background noise and cancelling it, through its reproduction by same audio system, after appropriate prior processing. One or more microphones can be used to pick up the noise in the passenger compartment of the automobile. It is evident that the signals picked up by the various microphones are correlated to one another, as they are picked up in the same noisy environment, i.e. from the same noise source.

Examples of these audio systems are given in US2003219137, WO02076148 and JP2009232204.

In any case, these systems are designed to suppress and not to produce the listener's perception of noise.

### STATE OF THE ART

Numerous studies have been conducted and published relating to the interaction between sound signals and brain activities.

Different techniques are known for generating sound that are capable of increasing the ability to communicate between cortical neural populations and of eliminating dysfunctional neural circuits.

A system for generating holophonic sound that produces appreciable results is described in EP0919109.

During trials with this system, it proved to have a surprising capacity for interaction with brain function, at the root of numerous psychological/psychiatric and neurological disorders.

The scientific articles—Aiello G., Finsterle G. (2009), “The use of psycho-acoustical transitional sessions in patients suffering of moderate and severe Alzheimer's disease.” ACTIVITAS NERVOSA SUPERIOR REDIVIVA, vol. 51, p. 36-45, ISSN: 1337-933X and Finsterle G. (2007), L'Incanto di Orfeo e i sincronismi neurali corticali. Introduzione alla Psico-Acustica Transizionale, in AA.VV., La condivisione del Benessere. II contributo della Psicologia Positiva, a cura di Delle Fave A., pp. 208-230, Franco Angeli, Milano, 2007, ISBN: 978-88-464-8490-1. Text adopted by the Faculty of Medicine of the State University of Milan, through instrumental and psychometric analysis, show the aforesaid capacity of interaction of the sound signals generated by the aforesaid system—described in EP0919109—with brain function.

However, this system provides for the use of four different sound sources, i.e. four acoustic diffusers.

### SUMMARY OF THE INVENTION

The object of the present invention is that of providing a sound generation system that is less expensive than the previous one and also portable.

The subject of the present invention is a sound generation system with two audio channels, in conformity with claim 1.

Further, the subject of the present invention is the coded signals obtained by means of the aforesaid sound generation system.

A further subject of the present invention is a storage means wherein said coded signals are stored.

The dependent claims describe preferred embodiments of the invention. The claims form an integral part of the present description.

### BRIEF DESCRIPTION OF THE FIGURES

Further features and advantages of the invention will be more apparent in the light of the detailed description of preferred, but not exclusive, embodiments of a sound generation system with two audio channels, shown by way of non-limiting example with the aid of FIG. 1, schematically representing said sound generation system with two audio channels.

FIG. 2 represents a block diagram of a preferred variant of the method implemented in/by the aforesaid system.

The parts with dashed lines are intended as optional.

The same reference numbers and letters in the figures identify the same element or components.

### DETAILED DESCRIPTION OF A PREFERRED EMBODIMENT OF THE INVENTION

The system according to the present invention, with reference to FIG. 1, comprises
means for generating two first audio signals, one for each audio channel Ch1, Ch2, wherein each first signal is a random noise, and wherein the two first signals are not correlated to each other and define a relative reference sound level,
means for adding a second mono or stereo audio signal to each of said first signals at a sound level having amplitude between +/−12 dB with respect to said relative reference sound level.

With only two channels it is possible to reproduce comparable effects obtainable by the aforesaid holophonic sound generation system—described in EP0919109—in the field of interaction with brain function.

The relative sound level is intended as being on average constant over time within +/−3 dB and/or being perceived as similar, in terms of sound level, by both ears. In the case of subjects with impaired hearing in one or both ears, the relative reference level of the two first signals can be appropriately corrected. This means that the system can be provided with appropriate means for compensating hearing defects of a subject.

Clearly, this also reflects on the sound level of the second signal.

Likewise, if a subject suffers from tinnitus, one or more filter notches can be provided and appropriately tuned.

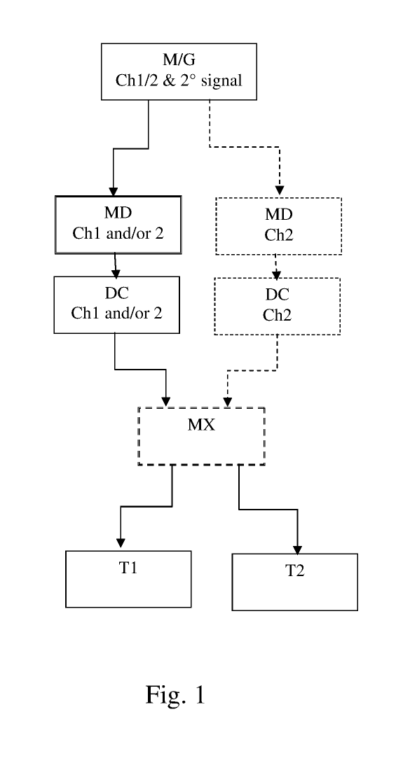

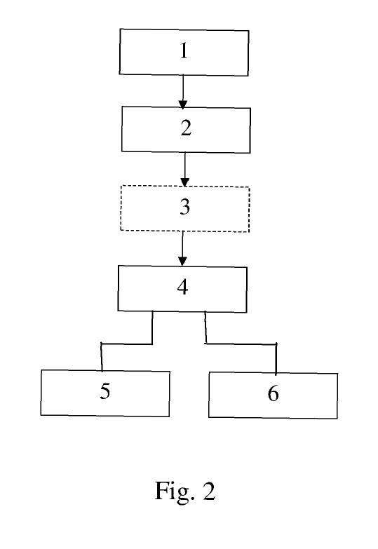

The steps performed by the system are described below (FIG. 2):
generating 1 two first audio signals, one for each audio channel Ch1, Ch2, wherein each first signal is a random noise, and wherein the two first signals are not correlated to each other and define a relative reference sound level, preferably with an amplitude that is constant over time (+/−3 dB), for example of pink noise type;
generating 2 any second audio signal having a sound level between +/−12 dB with respect to the aforesaid relative reference level and
adding/mixing 4 said second signal to/with each of said two first audio signals.

The signals obtained can be appropriately stored 5 and/or played 6.

It is preferable for the first signals to be of the same type, for example, both of the pink noise type, or both of the Gaussian type, etc.

Optionally, said first signals and/or said second signal are modulated 3 in amplitude within +/−12 dB with one or more frequencies in the range between 0.1 to 200 Hz.

According to a preferred variant of the invention the first signals and the second signal are not modulated in amplitude and the second signal is monophonic. While the first signals are perceived around the head, the second signal is perceived “in the middle” of the head.

According to a further preferred variant of the invention the first signals are not modulated in amplitude, while the second signal can be modulated in amplitude with frequencies in the range between 0.1-200 Hz and amplitude variable over time between +/−12 dB.

In particular, the signals modulated in amplitude can be modulated both according to a single frequency and according to several simultaneous and distinct modulating frequencies.

In any case, the relative sound level defined by said first signals is an average level over time.

According to a further preferred variant of the invention, both the first signals and the second signal are modulated in amplitude with different frequencies (between first signals and second signal), for example 0.5 Hz, 8 Hz, 40 Hz and with a ratio between maximum and minimum amplitude peak always between +/−12 dB with respect to the aforesaid average relative reference level.

Therefore, preferred modulation frequencies are in the range between 0.1-7.9 Hz (Delta and Theta brain frequency ranges) and/or in the range between 8-14 Hz (Alpha brain frequency range) and/or in the range between 30-42 Hz (Gamma brain frequency range).

Studies performed have identified the frequencies of around 0.5 and 8 Hz as the frequencies that trigger a “system reset” in the brain, considered innate and which can be triggered with the present invention, as was found by means of the aforesaid system with four channels.

A modulation with frequency of 40 Hz instead increases dopamine production in the brain.

A similar effect was recently obtained by irradiating the brains of mice with pulsed light, again at 40 Hz, after further manipulations.

The advantage obtained by the present system, which makes it possible to obtain similar results without cruelty, is evident.

Although a second signal is generically mentioned, it can either be monophonic, i.e. a single signal added/mixed in the same way to/with both the two first signals, or a stereophonic signal. These are two signals correlated to each other and added separately to the two first signals. One channel (of the stereo pair) added to one of the first signals and the other channel (of the stereo pair) added to the other of the two first signals.

The two signals obtained and destined to be played simultaneously, each on one of the channels Ch1 and Ch2, can be referred to generically as biphonic signal, as they are not correlated to each other like a stereophonic signal.

According to a preferred embodiment of the invention, with reference to FIG. 1, the playing system exploits a music playing device with two channels, comprising
means for storing audio tracks,
means for decoding said audio tracks DC and
only one pair of transducers that can be a known pair of loudspeakers or stereo headphones.

Optionally means for mixing MX with two channels and/or means for modulation MD of the audio tracks can be present, as described below.

According to a preferred variant of the system, both the first signals and the second signal are stored independently to one another as monophonic sound tracks and can be selected and modulated in frequency and amplitude in relation to specific needs. Moreover, the system comprises means for simultaneously playing said tracks related to the first signals and said track (optionally stereophonic) related to the second signal. In this case, with reference to FIG. 1, all the blocks and both the branches are present separately.

According to another preferred variant of the invention, the system comprises storage means wherein a plurality of first and second signals, already modulated in amplitude, in the form of audio tracks, are stored. Moreover, the system comprises means for simultaneously playing said tracks related to the first signals and a track related to said second signal as described above. In this case, with reference to FIG. 1, the blocks MD are not present and both branches are present separately.

According to a further preferred variant of the invention, the system comprises storage means for storing biphonic tracks resulting from previously adding said first signals to said second signal, optionally already modulated in amplitude according to one of the aforesaid frequencies. In this case, the dashed lines in FIG. 1 are not present, and the system is thus composed of only the parts M, DC, with two channels, and T1/T2. Moreover, as the audio tracks are obtained by means of the present method, it is also a part of the present invention, whether intended in terms of sound signals, electric signals or digital signals coded by means of any known coding.

The audio tracks can be coded in a known way, for example in MP3, WAW or AC3 format, etc. This means that the signals related to only two audio channels can be coded and stored separately or can be coded and stored as a pair of biphonic signals, as occurs for normal stereophonic music signals.

However, it is evident that these are not correlated signals as in the case of common stereo audio tracks of musical tracks.

The present invention can advantageously be implemented through a computer program that comprises coding means for performing one or more steps of the method, when this program is executed on a computer. Therefore, the scope of protection also extends to said computer program and also to computer readable media comprising program coding means for performing one or more steps of the method, when said program is executed on a computer.

Variants of embodiment to the non-limiting example described are possible, without departing from the scope of protection of the present invention, comprising all equivalent embodiments for those skilled in the art.

From the above description, a person skilled in the art is capable of implementing the object of the invention without adding further structural details. The elements and the features illustrated in the various preferred embodiments can be combined without however departing from the scope of protection of the present application.

1. A sound generation system with only two audio channels for treating psychological/psychiatric and neurological disorders, comprising
means for generating two first audio signals (G), one for each audio channel (Ch1, Ch2), wherein each first signal is a random noise, and wherein the two first signals are not correlated to each other and define a relative reference sound level,
means for generating and adding/mixing a second audio signal to/with each of said first signals at a sound level having amplitude between +/−12 dB with respect to said relative reference sound level.

2. The system according to claim 1, wherein said relative reference sound level is on average constant over time within +/−3 dB.

3. The system according to claim 1, further comprising means for modulating (MD) said first audio signals and/or said second audio signal in amplitude, within +/−12 dB, with one or more simultaneous amplitude modulations with frequency/frequencies in the range between 0.1-200 Hz.

4. The system according to claim 1, wherein said random noise is of the Pink, or White, or Gaussian type, or an equivalent thereof.

5. The system according to claim 3, wherein said amplitude modulation frequencies are in the range between 0.1-7.9 Hz and/or in the range between 8-14 Hz and/or in the range between 30-42 Hz.

6-14. (canceled)

15. The system according to claim 4, wherein said amplitude modulation frequencies are in the range between 0.1-7.9 Hz and/or in the range between 8-14 Hz and/or in the range between 30-42 Hz.

16. The system according to claim 1, comprising
means for storing (M) said first and second audio signals, and/or
means for mixing/adding (MX) said second signal with/to said first audio signals,
means for playing (T) the audio signals through said only two audio channels.

17. A method for the sound generation of only two audio signals for treating psychological/psychiatric and neurological disorders, comprising the following steps:
(1) generating two first audio signals, one for each audio channel (Ch1, Ch2), wherein each first signal is a random noise, and wherein the two first signals are not correlated to each other and define a relative reference sound level;
(2) generating any second audio signal having a sound level in the range between +/−12 dB with respect to the relative reference level, and
(4) adding/mixing said second signal to/with each of said two first audio signals.

18. The method according to claim 17, further comprising a step of storing and/or playing (5, 6) the only two audio signals obtained by the preceding steps (1, 2, 4).

19. The method according to claim 17, wherein said random noise is of the Pink, or White, or Gaussian type, etc., and/or wherein said first audio signals and/or said second audio signal is/are modulated in amplitude (3) before said adding/mixing step.

20. The method according to claim 17, further comprising said step (3) of modulating said first audio signals and/or said second audio signal in amplitude, within +/−12 dB, with one or more simultaneous amplitude modulations with frequency/frequencies in the range between 0.1-200 Hz.

21. The method according to claim 17, wherein said second signal comprises a stereo signal having two separate channels, each one of which is added/mixed to/with one of said first audio signals.

22. The method according to claim 18, wherein said second signal comprises a stereo signal having two separate channels, each one of which is added/mixed to/with one of said first audio signals.

23. The method according to claim 19, wherein said second signal comprises a stereo signal having two separate channels, each one of which is added/mixed to/with one of said first audio signals.

24. The method according to claim 20, wherein said second signal comprises a stereo signal having two separate channels, each one of which is added/mixed to/with one of said first audio signals.

25. Computer program comprising program coding means adapted to execute all the steps of claim 17, when said program is run on a computer.

26. A pair of electric signals or a coded biphonic audio signal related to only two separate channels (Ch1, Ch2) for treating psychological/psychiatric and neurological disorders, defined by
two first audio signals, one for each audio channel (Ch1, Ch2), wherein each first signal is a random noise, and wherein the two first signals are not correlated to each other and define a relative reference sound level;
any second audio signal having a sound level in the range between +/−12 dB with respect to the relative reference level, and added/mixed to/with said first audio signals.

27. Storage means wherein said biphonic audio signal as described in claim 26 is stored.
